(19) United States
(12) Patent Application Publication
(10) Pub. No.: US 2003/0175966 A1
(43) Pub. Date: Sep. 18, 2003

(54) Cationic sugar derivatives for gene transfer

(75) Inventors: Jinkang Wang, San Francisco, CA (US); Michael Bennett, Ei Sobrante, CA (US); Xuegong Wang, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Genteric, Inc., Alameda, CA

(21) Appl. No.: 10/100,466

(22) Filed: Mar. 14, 2002

(51) Int. Cl. C12Q 1/68; A61K 48/00; C12N 15/85

(52) U.S. Cl. 435/455; 536/55; 514/44; 435/6

(57) ABSTRACT

This present invention provides a class of gene transfection reagents, which have a structure containing a nucleic acid binding domain and sugar targeting domain. The compounds are easy to synthesize and formulate. The formulated compound associates with DNA to form small particles with nearly neutral surface charge. The sugar domain plays a role as a tissue target ligand located on the surface of the nucleic acid complex, which promotes the receptor-mediated gene transfection. In the presence of proteins, these DNA complexes do not bind with proteins to form precipitates. The complexes are also stable when stored at 4° C. for a long time.

### BACKGROUND OF THE INVENTION

Many systems for administering active substances into cells are already known, such as liposomes, nanoparticles, polymer particles, immuno- and ligand-complexes and cyclodextrins (see, Drug Transport in antimicrobial and anticancer chemotherapy. G. Papadakou Ed., CRC Press, 1995). However, none of these systems has proved to be truly satisfactory for the in vivo transport of nucleic acids such as, for example, deoxyribonucleic acid (DNA).

Satisfactory in vivo transport of nucleic acids into cells is necessary for example, in gene therapy. Gene transfer is the transfection of a nucleic acid-based product, such as a gene, into the cells of an organism. The gene is expressed in the cells after it has been introduced into the organism. Several methods of cell transfection exist at present. These methods include for example, use of calcium phosphate, microinjection, protoplasmic fusion; electroporation and injection of free DNA; viral infection; and synthetic vectors.

Gene delivery systems play an important role in human gene therapy. The foreign genes are required to be delivered into the target cells, and enter the nucleus for transcription and expression. Viral vector gene delivery systems have showed therapeutic level of gene expression and efficacy in animals and human clinical trials. Several kinds of viruses, including retrovirus, adenovirus, adeno-associated virus (AAV), and herpes simplex virus (HSV), have been manipulated for use in gene transfer and gene therapy applications. As different viral vector systems have their own unique advantages and disadvantages, they each have applications for which they are best suited. However, recent experiences with viral transfer of genes have shown the possible deleterious effects of viral gene delivery including inflammation of the meninges and potentially fatal reactions by the patient's immune system.

The processes to prepare viral vector gene delivery systems are also complicated and not suitable to operate. Therefore, non-viral gene delivery systems have been extremely attractive and extensively investigated in the last 15 years. A number of lipid, peptide and polymer-based vectors have been designed. These delivery vectors show good transfection efficiency in cell cultures and the preparation methods are much easier than the viral delivery vectors. Cationic lipids show very good gene transfection in the lung. Some small molecules show enhancement in gene transfection in muscle. However, in vivo gene transfer is complicated by biological fluid interactions, immune clearance, toxicity and biodistribution, depending on the route of administration. Most of these non-viral gene carriers show poor in vivo gene expression, high toxicity and poor storage stability. In most cases, these vectors form DNA complex particles with negatively charged surface and usually show poor transfection activity, and the complexes with positive surface charge would bind with proteins in biological fluid to form big particles, or are even precipitated. This also decreases the biodistribution and transfection efficiency.

There is increasing interest in the use of synthetic vectors, such as lipid or polypeptide vectors. Synthetic vectors appear to be less toxic than the viral vectors. Among synthetic vectors, lipid vectors, such as liposomes, appear to have the advantage over polypeptide vectors of being potentially less immunogenic and, for the time being, more efficient. However, the use of conventional liposomes for DNA delivery is very limited because of the low encapsulation rate and their inability to compact large molecules, such as nucleic acids.

The formation of DNA complexes with cationic lipids has been studied by various laboratories (see, Felgner et al., PNAS 84, 7413-7417 (1987); Gao et al., Biochem. Biophys. Res. Commun. 179, 280-285, (1991); Behr, Bioconj. Chem. 5, 382-389 (1994)). These DNA-cationic lipid complexes have also been designated in the past using the term lipoplexes (see, P. Felgner et al., Hum. Genet. Ther., 8, 511-512, 1997). Cationic lipids enable the formation of relatively stable electrostatic complexes with DNA, which is a poylanionic substance.

The use of cationic lipids has been shown to be effective in the transport of DNA in cell culture. However, the in vivo application of these complexes for gene transfer, particularly after systemic administration, is poorly documented (see, Zhu et al., Science 261, 209-211 (1993); Thierry et al., PNAS 92, 9742-9746 (1995); Hofland et al., PNAS 93, 7305-7309 (1996)).

Cationized polymers have also been investigated as vector complexes for transfecting DNA. For example, vectors called “neutraplexes” containing a cationic polysaccaride or oligosaccharide matrix have been described in U.S. application Ser. No. 09/126,402. Such vectors also contain an amphiphilic compound, such as a lipid.

Chitosan conjugates having pendant galactose residues have also been investigated as a gene delivery vector. See Murata et al., “Possibility of Application of Quaternary Chitosan Having Pendant Galactose Residues as Gene Delivery Tool,” Carbohydrate Polymers, 29(1):69-74 (1996); Murata et al., “Design of Quaternary Chitosan Conjugate Having Antennary Galactose Residues as a Gene Delivery Tool,” Carbohydrate Polymers, 32:105-109 (1997). Chitosan is a biodegradable cationic natural polysaccharide. Due to its good biocompatibility and toxicity profile, it has been widely used in pharmaceutical research and industry as a carrier for drugs and gene delivery. However, because its performance is rather restricted to the gastrointestinal area, it has limited use in vivo.

Galactosylated polyethyleneimine/DNA complexes have also been investigated. See Bettinger, et al., “Size Reduction of Galactosylated PEI/DNA Complexes Improves Lectin-Mediated Gene Transfer into Hepatocytes,” Bioconjugate Chem., 10:558-561 (1999). Although the mechanism underlying these complexes has been elucidated in vitro, it is uncertain whether this can be extended to in vivo applications.

Therefore, there is a need for an improved vector for administering a nucleic acid molecule into a cell. The present invention fulfills this and other needs.

### BRIEF SUMMARY OF THE INVENTION

The present invention relates to design, synthesis, and formulation of non-viral gene delivery reagents for transferring nucleic acid (e.g., plasmid DNA) to cells. The reagents include molecules containing a nucleic acid binding domain and sugar targeting domain. The transfected cells include in vitro and in vivo. The delivery reagents of the present invention are different from cationic lipids, peptides, and polymers. The delivery carriers are able to protect nucleic acid (e.g., DNA) from degradation, afford opportunities to target cells of therapeutic interest, and enhance gene transfection efficiency. Advantageously, the delivery system also does not interact with proteins in biological fluids to aggregate or precipitate. The system is very simple to operate, and has a long storage stability time.

As such, in one embodiment, the present invention provides compounds of Formula I:

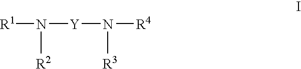

In Formula I, R¹ is a C₃-C₂₀ carbohydrate with an optional linker. R², in Formula I, is a group selected from a hydrogen, an alkyl group, and a boronic acid group. Y, in Formula I, is an optionally substituted alkylene group or (CH₂CH₂O)ₘ, wherein m is about 2 to about 80. In Formula I, R³ is selected from the group of a hydrogen, an alkyl group, and a cationic moiety. R⁴, in Formula I, is selected from the group of an alkyl group, and a cationic moiety. In an alternative embodiment, R³ and R⁴ and the nitrogens to which they are attached, join together to form an optionally substituted five- or six-membered carbocyclic or heterocylic ring. In a preferred embodiment, the cationic moiety is a quaternary nitrogen functional group.

In preferred aspects, compounds of Formula I have the structure of Formula Ia:

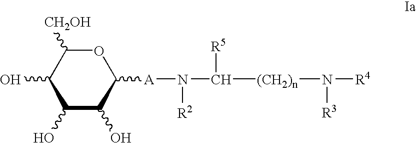

In Formula Ia, A is a linker or a C₆-C₁₂ carbohydrate with an optional linker. In Formula Ia, R² is selected from the group of a hydrogen, an alkyl group, and a boronic acid group. In Formula Ia, n is an integer from 1-5 inclusive. R³, in Formula Ia, is selected from the group of a hydrogen, an alkyl group, and a cationic moiety. In Formula Ia, R⁴ is selected from the group of an alkyl group, or a cationic moiety. In an alternative embodiment, R³ and R⁴ and the nitrogens to which they are attached, join together to form an optionally substituted five- or six-membered carbocyclic or heterocylic ring.

In certain other preferred aspects, the compounds of Formula I have the structure of Formula Ib:

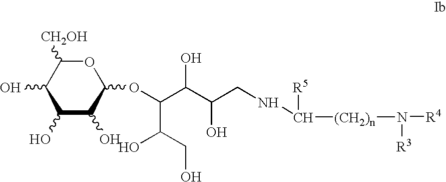

In Formula Ib, R³ is selected from the group of a hydrogen, an alkyl group, and a cationic moiety. In Formula Ib, R⁴ is selected from the group of an alkyl group, a cationic moiety. In Formula Ib, R⁵ is selected from the group of a hydrogen, a carboxyl group and an alkyl group. In Formula Ib, n is an integer from 2-3 inclusive.

In an alternative embodiment, R³ and R⁴ and the nitrogens to which they are attached, join together to form an optionally substituted five- or six-membered carbocyclic or heterocylic ring

In yet another embodiment, the present invention provides a transfection complex comprising a nucleic acid and a compound having Formula I.

In still yet another embodiment, the present invention provides a method for transfecting mammalian cells, the method comprising contacting a nucleic acid with a compound having Formula I.

These and other aspects will become more apparent when read with the accompanying drawings and the detailed description which follow.

### BRIEF DESCRIPTION OF THE DRAWINGS

FIG. 1 provides a schematic of compounds according to one embodiment of the present invention.

FIGS. 2A-B provide schematics of reaction to produce a compound of the invention.

FIGS. 3A-B provide a list of chemical structures of compounds according to one embodiment of the present invention.

FIG. 4 is a bar graph illustrating the hGH gene expression with DNA/S-5 complexes in rat SMG.

FIG. 5 is a bar graph illustrating the Luciferase gene expression in mouse intestine.

FIG. 6 is a bar graph illustrating the Luciferase gene expression in CHO cells.

### DETAILED DESCRIPTION OF THE INVENTION AND PREFERRED EMBODIMENTS

As used herein, the term “alkyl” denotes branched or unbranched hydrocarbon chains, preferably having about 1 to about 18 carbons, such as, methyl, ethyl, n-propyl, iso-propyl, n-butyl, sec-butyl, iso-butyl, tert-butyl, octa-decyl and 2-methylpentyl. These groups can be optionally substituted with one or more functional groups which are attached commonly to such chains, such as, hydroxyl, bromo, fluoro, chloro, iodo, mercapto or thio, cyano, alkylthio, heterocyclyl, aryl, heteroaryl, carboxyl, carbalkoyl, alkyl, alkenyl, nitro, amino, alkoxyl, amido, and the like to form alkyl groups such as trifluoro methyl, 3-hydroxyhexyl, 2-carboxypropyl, 2-fluoroethyl, carboxymethyl, cyanobutyl and the like.

The term “alkylene” refers to a divalent alkyl group as defined above, such as methylene (—CH₂—), propylene (—CH₂CH₂CH₂—), chloroethylene (—CHClCH₂—), 2-thiobutene —CH₂CH(SH)CH₂CH₂, 1-bromo-3-hydroxyl-4-methylpentene (—CHBrCH₂CH(OH)CH(CH₃)CH₂—), and the like.

The term “alkenyl” denotes branched or unbranched hydrocarbon chains containing one or more carbon-carbon double bonds.

The term “alkynyl” refers to branched or unbranched hydrocarbon chains containing one or more carbon-carbon triple bonds.

The term “aryl” denotes a chain of carbon atoms which form at least one aromatic ring having preferably between about 6-14 carbon atoms, such as phenyl, naphthyl, and the like, and which may be substituted with one or more functional groups which are attached commonly to such chains, such as hydroxyl, bromo, fluoro, chloro, iodo, mercapto or thio, cyano, cyanoamido, alkylthio, heterocycle, aryl, heteroaryl, carboxyl, carbalkoyl, alkyl, alkenyl, nitro, amino, alkoxyl, amido, and the like to form aryl groups such as biphenyl, iodobiphenyl, methoxybiphenyl, anthryl, bromophenyl, iodophenyl, chlorophenyl, hydroxyphenyl, methoxyphenyl, formylphenyl, acetylphenyl, trifluoromethylthiophenyl, trifluoromethoxyphenyl, alkylthiophenyl, trialkylammoniumphenyl, amidophenyl, thiazolylphenyl, oxazolylphenyl, imidazolylphenyl, imidazolyhnethylphenyl, and the like.

The term “acyl” denotes the —C(O)R group, wherein R is alkyl or aryl as defined above, such as formyl, acetyl, propionyl, or butyryl.

The term “alkoxy” denotes —OR—, wherein R is alkyl.

The term “amido” denotes an amide linkage: —C(O)NR— (wherein R is hydrogen or alkyl).

The term “amino” denotes an amine linkage: —NR—, wherein R is hydrogen or alkyl.

The term “carboxyl” denotes —C(O)O—, and the term “carbonyl” denotes —C(O)—.

The term “carbonate” indicates —OC(O)O—.

The term “carbamate” denotes —NHC(O)O—, and the term “urea” denotes —NHC(O)NH—.

The term “nucleic acid” refers to a polymer containing at least two nucleotides. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleotides are the monomeric units of nucleic acid polymers. A “polynucleotide” is distinguished here from an “oligonucleotide” by containing more than 80 monomeric units; oligonucleotides contain from 2 to 80 nucleotides. The term nucleic acid includes deoxyribonucleic acid (DNA) and ribonucleic acid (RNA). The term encompasses sequences that include any of the known base analogs of DNA and RNA.

DNA may be in the form of anti-sense, plasmid DNA, parts of a plasmid DNA, product of a polymerase chain reaction (PCR), vectors (P1, PAC, BAC, YAC, artificial chromosomes), expression cassettes, chimeric sequences, chromosomal DNA, or derivatives of these groups. RNA may be in the form of oligonucleotide RNA, tRNA (transfer RNA), snRNA (small nuclear RNA), rRNA (ribosomal RNA), mRNA (messenger RNA), anti-sense RNA, ribozymes, chimeric sequences, or derivatives of these groups.

“Antisense” is a polynucleotide that interferes with the function of DNA and/or RNA. This may result in suppression of expression. Natural nucleic acids have a phosphate backbone, artificial nucleic acids may contain other types of backbones and bases. These include PNAs (peptide nucleic acids), phosphothionates, and other variants of the phosphate backbone of native nucleic acids. In addition, DNA and RNA may be single, double, triple, or quadruple stranded.

The term “recombinant DNA molecule” as used herein refers to a DNA molecule that is comprised of segments of DNA joined together by means of molecular biological techniques. “Expression cassette” refers to a natural or recombinantly produced polynucleotide molecule that is capable of expressing protein(s). A DNA expression cassette typically includes a promoter (allowing transcription initiation), and a sequence encoding one or more proteins. Optionally, the expression cassette may include trancriptional enhancers, noncoding sequences, splicing signals, transcription termination signals, and polyadenylation signals. An RNA expression cassette typically includes a translation initiation codon (allowing translation initiation), and a sequence encoding one or more proteins. Optionally, the expression cassette may include translation termination signals, a polyadenosine sequence, internal ribosome entry sites (IRES), and non-coding sequences.

The term “gene” refers to a nucleic acid (e.g., DNA) sequence that comprises coding sequences necessary for the production of a polypeptide or precursor (e.g., myosin heavy chain). The polypeptide can be encoded by a full length coding sequence or by any portion of the coding sequence so long as the desired activity or functional properties (e.g., enzymatic activity, ligand binding, signal transduction, and the like) of the full-length or fragment are retained. The term also encompasses the coding region of a structural gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ non-translated sequences. The sequences that are located 3′ or downstream of the coding region and which are present on the mRNA are referred to as 3′ nontranslated sequences. The term “gene” encompasses both cDNA and genomic forms of a gene. A genomic form or clone of a gene contains the coding region interrupted with noncoding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Upregulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “linker” means a molecule that joins the carbohydrate targeting domain with the nucleic acid binding domain. In certain aspects, the sugar targeting domain and the nucleic acid binding domain are physically linked by, for example, covalent chemical bonds, physical forces such van der Waals or hydrophobic interactions. In certain preferred embodiments, the linker is an ethylene oxide linker having the structure (CH₂CH₂O)ₘ, wherein m is about 2 to about 80.

In another embodiment, the linker is a linking pair. In certain aspects, the “linking pair” refers to a first molecule (A) and a second molecule (B) (e.g., A-B) that specifically bind to each other. In other aspects, the sugar targeting domain terminates in a reactive group, such as a carboxylic acid group, a thiol group or an amine group. The nucleic acid binding domain ends in a complementary functional group. By way of the example only, a carboxylic acid on the carbohydrate targeting moiety may react with an amine of the nucleic acid binding domain to form an amide coupling.

In another embodiment, exemplary linking pairs include, but are not limited to, any haptenic or antigenic compound in combination with a corresponding antibody or binding portion or fragment thereof (e.g., digoxigenin and anti-digoxigenin; mouse immunoglobulin and goat anti-mouse immunoglobulin) and nonimmunological binding pairs (e.g., biotin-avidin, biotin-streptavidin and the like).

In certain aspects, A and B form a linkage such as (in either orientation): —C(O)O—, —C(O)NH—, —OC(O)NH—, —S—S—, —C(S)O—, —C(S)NH—, —NHC(O)NH—, —SO₂NH—, —SONH—, phosphate, phosphonate and phosphinate. In each of the groups provided above, NH is shown for brevity, but each of the linkages can contain substituted (e.g., N-alkyl or N-acyl) linkages as well.

The term “boronic acid group” means a —B(OH)₂ group.

The term “cationic moiety” means a group having a net positive charge such as a quaternary nitrogen. Other examples, include, but are not limited to, primary amines, secondary amines, tertiary amines, quaternary amines, quanidine, and the like. Those of skill in the art will appreciate that any functional group that provides a positive charge at biological pH (such as about pH 7.4) are suitable for use in the present invention.

As used herein, the term carbohydrate moiety means a functional compound having the formula Cₙ(H₂O)ₙ wherein at least one carbon is reduced such that an oxygen is removed and a hydrogen is added. The carbohydrate moiety is between 3 and 20 carbons in length. For example, in an aldose, the aldehydic carbon is reduced to a methylene group and attached to a nitrogen functional group. Suitable carbohydrate groups include all reducing sugars, which can be reductively aminated with a compound having a primary amino group. Suitable carbohydrates include, but are not limited to, aldoses, ketoses, monosaccharides, disaccharides, trisaccharides and polysaccharides wherein at least one carbon is modified to be attached to a functional group such as to a nitrogen functional group or a linker. Suitable monosaccharides include, but are not limited to, glucose, fructose, ribose, galactose, mannose, arabinose. Suitable disaccharides include, but are not limited to lactose, cellobiose, gentibiose, and maltose. In each of the foregoing, at least one carbon is modified such that it can be attached to a functional group.

### I. GENERAL

In certain embodiments, the present invention provides a class of gene transfection reagents, which possess a nucleic acid binding domain and a sugar targeting domain. The compounds are easy to synthesize and formulate. In certain aspects, the formulated compound associate with nucleic acid to form small particles with nearly neutral surface charge. In certain preferred aspects, the sugar domain plays a role as a tissue target ligand located on the surface of the nucleic acid complex, which also promotes receptor-mediated gene transfection. Advantageously, in the presence of proteins, these nucleic complexes do not bind with proteins to form aggregated particles or precipitates. The complexes are also stable when stored at 4° C. for long periods of time.

### II. COMPOUNDS AND SYNTHESIS

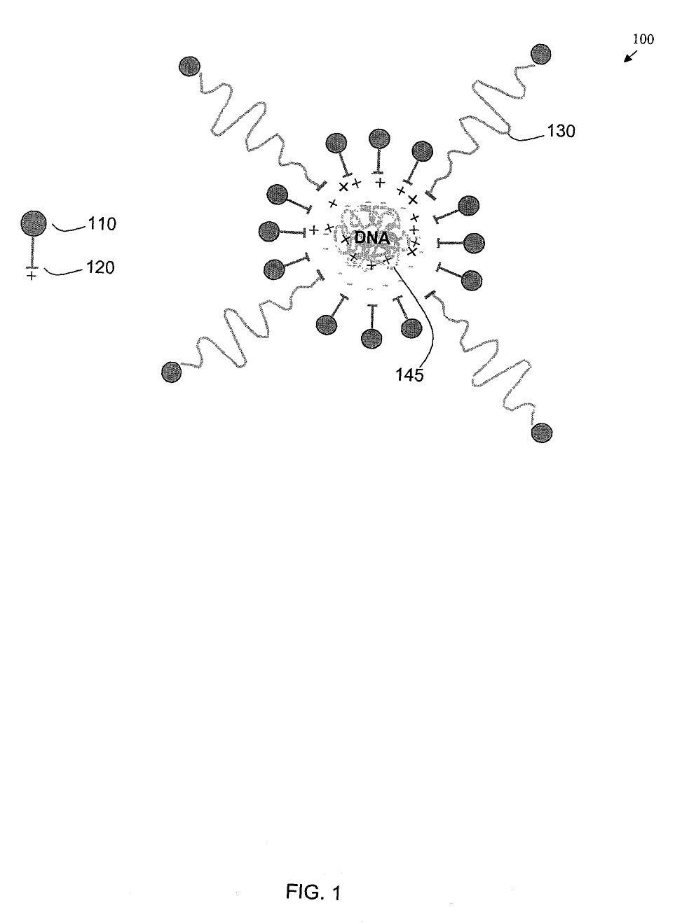

In certain embodiments, the present invention provides a class of gene transfection reagents, which have a structure containing a nucleic acid binding domain and a sugar targeting domain. FIG. 1 is an example of a representative transfection reagent of the present invention. This structure is merely an illustration and should not limit the scope of the claims herein. One of ordinary skill in the art will recognize other variations, modifications, and alternatives.

In one embodiment, the compounds of the present invention have a carbohydrate or sugar targeting domain 110 and a nucleic acid binding domain 120. In general, the nucleic acid binding domain 120 is positively charged in order to bind to the negatively charged phosphates of nucleic acid 145 (e.g., DNA). The sugar targeting domain 110 is designed to target or bind to carbohydrate binding surfaces of cells. In certain preferred embodiments, the carbohydrate targeting domain 110 can be specific for glycoprotein receptors of cells. In certain instances, these glycoprotein receptor sites are attached to a specific tissue, such as an organ. Organs include, but are not limited to, the heart, spleen, lung, kidney and liver.

In certain other instances, the carbohydrate or sugar targeting domain 110 is recognized by endogenous lectins mediating critical cellular functions. In other embodiments, the carbohydrate or sugar targeting domain 110 functions in protein synthesis, confers protein stability or resistance to degradation, regulates intracellular signaling, and the like. In certain other instances, the carbohydrate or sugar targeting domain 110 regulates lateral mobility of proteins in plasma membranes, controls protein-protein interaction, and mediates organization of proteins in domains or lattices on the cell surface. In certain embodiments, the carbohydrate domain is specific for a target tissue.

In certain aspects, there is a linker 130 between the sugar targeting domain 110 and the nucleic acid binding domain 120. The linker can be of various sizes and lengths. In certain preferred embodiments, the linker is an ethylene oxide linker having the structure —(CH₂CH₂O)ₘ, wherein m is about 2 to about 80.

In certain instances, the compounds of the present invention “coat” the nucleic acid. The nucleic acid can be coated the compounds of the present invention using various ratios of cationic moieties (e.g., amine groups on the sugar domain) to phosphate groups on the nucleic acid binding domain. In certain embodiments, this ratio between the cationic groups (positive charges): to the phosphate groups (negative charges) is about 1-512:1. In a preferred embodiment, this ratio is about 128-256:1.

In certain preferred embodiments, the present invention provides compounds having Formula I:

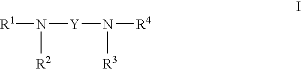

wherein R¹, R², R³ and R⁴ have been described above. The compounds of the present invention can be made by a variety of techniques, by using commercially available starting materials. The compounds of the present invention are useful as transfection reagents.

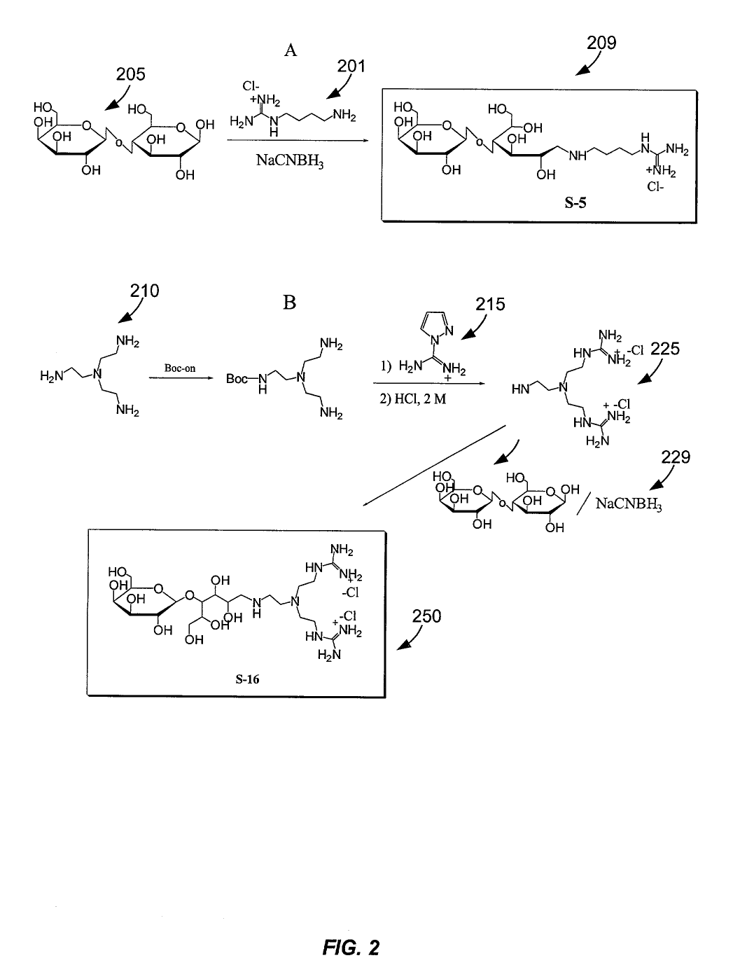

FIGS. 2A-B are examples of a representative synthetic schemes suitable for use in making the compounds of the present invention. These schemes are merely an illustration and should not limit the scope of the claims herein. One of ordinary skill in the art will recognize other variations, modifications, and alternatives.

As set forth in FIG. 2A, a mixture of agmatine sulfate 201, sodium cyanoborohydride 203 and lactose 205 are reacted in water under argon in a sealed tube to generate a compound of Formula I (S-5) 209. The reaction mixture can be purified by an anionic exchange column followed by several times recrystallization from water and methanol. The methanol can be removed by a rotary evaporator, and the water can be removed by lyophilization to give the product as a white solid.

In a second representative example as shown in FIG. 2B, tris(2-aminoethyl)amine 210 is partially protected with Boc-on. The two unprotected amino groups are treated with 1H-pyrazole-1-carboxamidine hydrochloride 215 to give the guanylated intermediate 225. The intermediate, sodium cyanoborohydride 229 and lactose 233 are reacted in water under argon in a sealed tube to generate a compound of Formula II (S-16) 250. The reaction mixture can be purified by an anionic exchange column followed by several times recrystallization from water and methanol. The methanol can be removed by a rotary evaporator, and the water can be removed by lyophilization to give the product as a white solid.

In certain preferred aspects, the compounds of Formula I have the structure of Formula Ia:

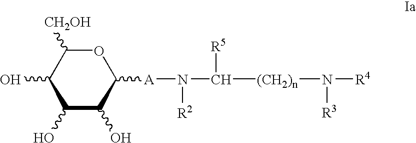

In Formula Ia, A is a linker or a C₆-C₁₂ carbohydrate with an optional linker. In one preferred aspect, the linker is a ethylene oxide having the structure —(CH₂CH₂O)ₘ, wherein m is about 2 to about 80. R² is selected from the group of a hydrogen, an alkyl group, and a boronic acid group. In Formula Ia, n is an integer from 1-5 inclusive. R³ is selected from the group of a hydrogen, an alkyl group, and a cationic moiety. R⁴ is selected from the group of an alkyl group, a cationic moiety. In an alternative embodiment, R³ and R⁴ and the nitrogens to which they are attached, join together to form an optionally substituted five- or six-membered carbocyclic or heterocylic ring.

In certain preferred aspects, the compounds of Formula I have the structure Ib:

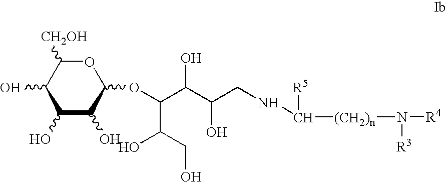

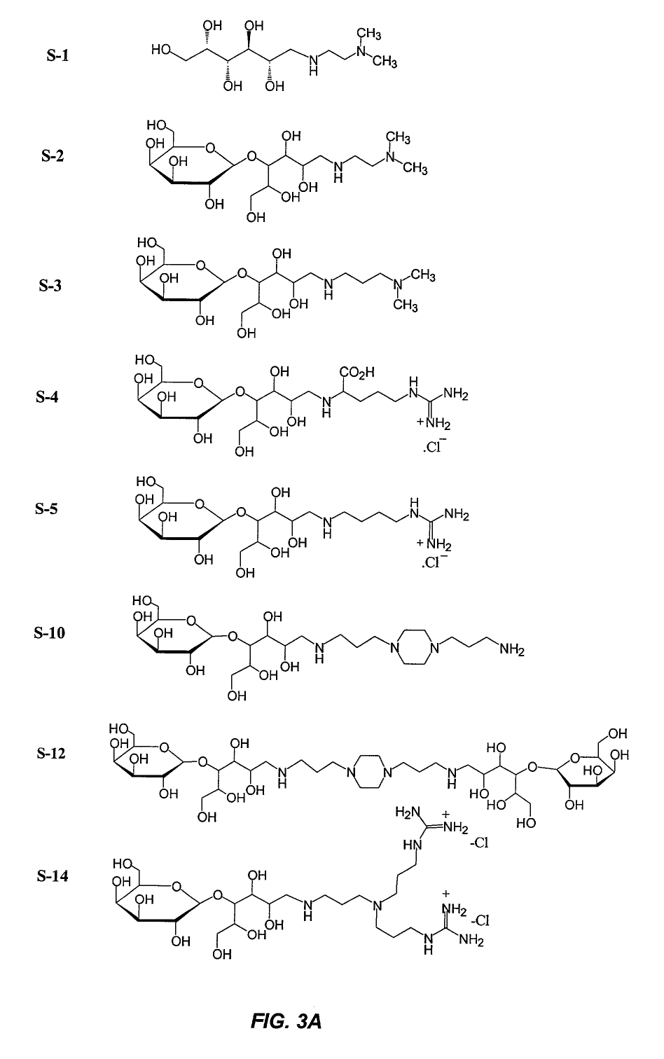

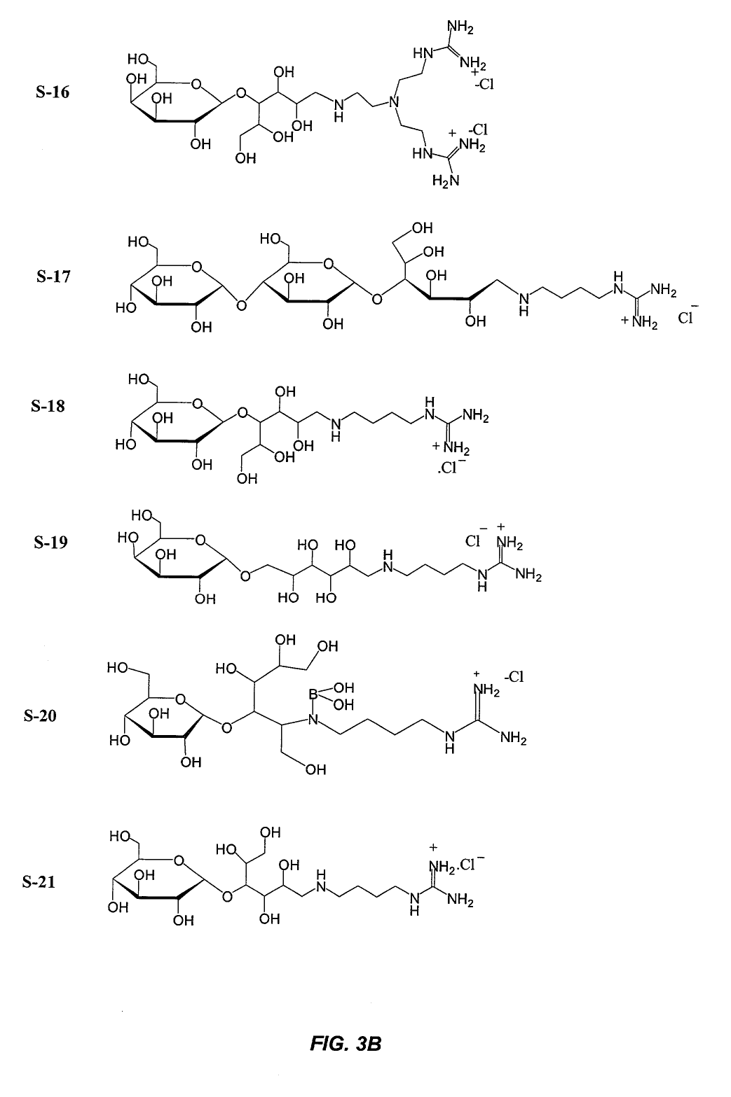

In Formula Ib, R³ is selected from a hydrogen, an alkyl group, and a cationic moiety. R⁴ is selected from an alkyl group, a cationic moiety. In an alternative embodiment, R³ and R⁴ and the nitrogens to which they are attached, join together to form an optionally substituted five- or six-membered carbocyclic or heterocylic ring. R⁵ is a member selected from the group consisting of a hydrogen, a carboxyl group and an alkyl group. In Formula Ib, n is an integer from 2 to 3 inclusive. Table I in FIGS. 3A-B sets forth preferred compounds of Formula I.

### III. DELIVERY OF NUCLEIC ACIDS

The process of delivering a polynucleotide to a cell has been commonly termed “transfection” or the process of “transfecting” and also it has been termed “transformation”. The polynucleotide can be used to produce a change in a cell that can be therapeutic. The delivery of polynucleotides or genetic material for therapeutic and research purposes is commonly called “gene therapy”. Nucleic acids of all types can be associated with the cationic lipids and liposomes of the present invention and subsequently can be transfected. These include DNA, RNA, DNA/RNA hybrids (each of which may be single or double stranded), including oligonucleotides such as antisense oligonucleotides, chimeric DNA-RNA polymers, and ribozymes, as well as modified versions of these nucleic acids wherein the modification may be in the base, the sugar moiety, the phosphate linkage, or in any combination thereof.

The nucleic acids can comprise an essential gene or fragment thereof, in which the target cell or cells is deficient in some manner. This can occur where the gene is lacking or where the gene is mutated resulting in under- or over-expression. The nucleic acids can also comprise antisense oligonucleotides. Such antisense oligonucleotides can be constructed to inhibit expression of a target gene. The foregoing are examples of nucleic acids that can be used with the present invention, and should not be construed to limit the invention in any way. Those skilled in the art will appreciate that other nucleic acids will be suitable for use in the present invention as well.

The delivery of nucleic acid can lead to modification of the DNA sequence of the target cell. The polynucleotides or genetic material being delivered are generally mixed with transfection reagents prior to delivery. The term “transfection” as used herein refers to the introduction of foreign DNA into eukaryotic cells. Transfection can be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics. The term “stable transfection” or “stably transfected” refers to the introduction and integration of foreign DNA into the genome of the transfected cell. The term “stable transfectant” refers to a cell which has stably integrated foreign DNA into the genomic DNA.

A “transfection reagent” or “delivery vehicle” is a compound or compounds that bind(s) to or complex(es) with oligonucleotides, polynucleotides, or other desired compounds and mediates their entry into cells. Examples of transfection reagents include, but are not limited to, cationic liposomes and lipids, polyamines, calcium phosphate precipitates, histone proteins, polyethylenimine, and polylysine complexes (polyethylenimine and polylysine are both toxic). Typically, when used for the delivery of nucleic acids, the transfection reagent has a net positive charge that binds to the polynucleotide's negative charge. For example, cationic liposomes or polylysine complexes have net positive charges that enable them to bind to DNA or RNA.

### IV. SPECIFIC TARGET TISSUES

Specific targeting moieties can be used with the complexes of this invention to target specific cells or tissues. In one embodiment, the targeting moiety, such as an antibody or antibody fragment, is attached to a hydrophilic polymer and is combined with the lipid:nucleic acid complex after complex formation. Thus, the use of a targeting moiety in combination with a complex provides the ability to conveniently customize the complex for delivery to specific cells and tissues.

Examples of effectors in lipid:nucleic acid complexes include nucleic acids encoding cytotoxins (e.g., diphtheria toxin (DT), Pseudomonas exotoxin A (PE), pertussis toxin (PT), and the pertussis adenylate cyclase (CYA)), antisense nucleic acid, ribozymes, labeled nucleic acids, and nucleic acids encoding tumor suppressor genes such as p53, p110Rb, and p72. These effectors can be specifically targeted to cells such as cancer cells, immune cells (e.g., B and T cells), and other desired cellular targets with a targeting moiety. For example, as described above, many cancers are characterized by overexpression of cell surface markers such as HER2, which is expressed in breast cancer cells, or IL17R, which is expressed in gliomas. Targeting moieties such as anti-HER2 and anti-IL17R antibodies or antibody fragments are used to deliver the lipid:nucleic acid complex to the cell of choice. The effector molecule is thus delivered to the specific cell type, providing a useful and specific therapeutic treatment.

### V. DISEASE TREATMENT

In yet another aspect of the invention comprises novel methods of treating diseases arising from infection by a pathogen or from an endogenous DNA deficiency. These methods comprise administering a nucleic acid aggregate and/or drug aggregate solution to a mammal suffering from a pathogenic infection or DNA deficiency. If the disease is the result of infection by a pathogen, the nucleic acid can be an antisense oligonucleotide targeted against an DNA sequence in the pathogen that is essential for development, metabolism, or reproduction of the pathogen. If the disease is a DNA deficiency (i.e., wherein certain endogenous DNA is missing or has been mutated), resulting in under- or over-expression, the nucleic acid maybe the normal DNA sequence.

The complex can be delivered by retrograde introduction into the submandibular glands. The complex can also be delivered either orally, or by direct administration to the lumen of the intestine. The retrograde delivery is by for example, surgical cannulation to a chosen organ duct, and injection of the DNA complex against the natural direction flow with a syringe, pump or the like. Oral delivery, also called gavage in animal models, is injection of DNA complexes in the gastrointestinal system by inserting a feeding tube into the stomach. Alternatively, the complex may be administered using enteric release capsules. Direct administration is by laparotomy, such as to inject the DNA complex directly into the lumen of the intestine.

### VI. EXAMPLES

This invention can be archived by the following examples. The examples and embodiments described herein and for illustrative purposes only, and various modifications will be apparent to those of skill in the art, the invention to be limited only by the scope of the claims.

### A. Example 1

Synthesis of Cationic Galactose Derivative (S-5)

The mixture of agmatine sulfate 0.77 g (3.38 mmol), sodium cyanoborohydride (5 M, 1 mL, 5 mmol), and lactose (2.4 g, 6.76 mmol) in water (4 mL) at pH 7 under argon in a sealed tube was stirred at 40° C. for 48 h. The reaction mixture was purified by an anionic exchange column followed by several times recrystallization from H₂O-MeOH. The methanol was removed by a rotary evaporator, and the water was removed by lyophilization to give the product as a white solid. ¹H NMR (D₂O, 300 MHz): δ 4.49 (1 H, dd,ƒ=5.7, 20.4), 4.40 and 4.32 (1 H, 2 broad s), 4.24 (1 H, broad m), 4.11 (1 H, broad m), 3.97-3.84 (3 H, m), 3.79-3.63 (8 H, m), 3.56-3.20 (1 H, m), 3.24 (3 H, m), 3.05 (2 H, m), 2.84 (1 H, m), 1.66 (4 H, broad m). HRMS (FAB): Calcd 457.2510 for C₁₇H₃₇N₄O₁₀, found 457.2504.

### B. Example 2

Protocol for Gene Transfer in Rat SMG

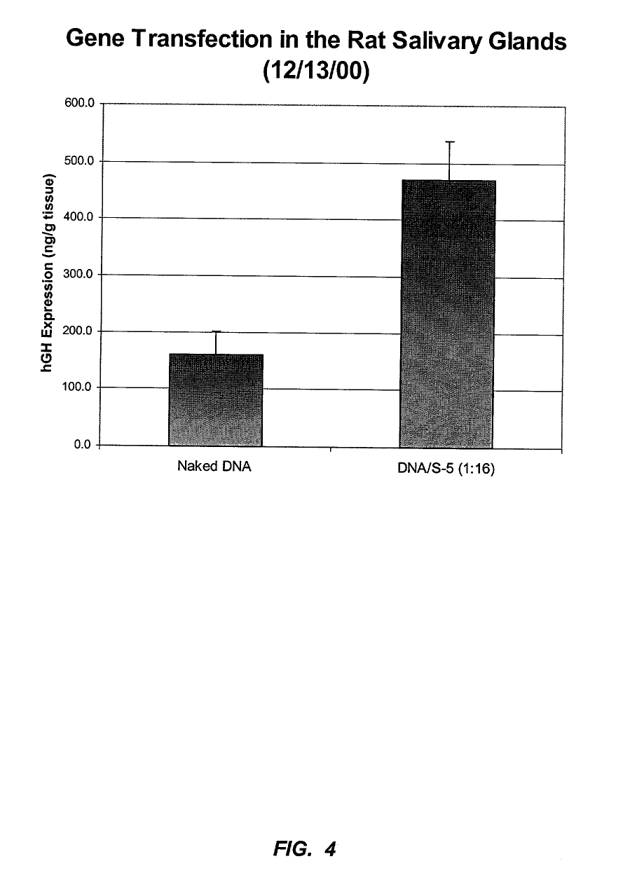

Male Sprague-Dawley rats (weighted 260-280 g) were fasted overnight prior to treatment. After administration of the anesthesia (i.m. injection of mixture of ketamine:xylazine:aceproamzine 30:6:1, mg/kg b. wt.), both right and left salivary gland ducts were cannulated with a modified polyethylene tubing (i.d. 0.005″) and cemented in place with a small drop of krazyglue. Atropine was then administrated subcutaneously (0.5 mg/kg b. wt.) and, after 10 min, 200 μl of the DNA-containing solution was then injected by retrograde induction. The tubing was kept in place for 10 additional min. The tubing was then gently removed. After 7 days, the rats were anesthetized by i.p administration of pentobarbital (50 mg/kg b. wt.). The right and left submandibular glands were then removed, and the tissues were homogenized in cold lysis buffer (0.1 mL buffer per 0.1 g tissue). The gene expression was determined by ELISA method, and is set forth in FIG. 4.

### C. Example 3

Protocol for Gene Transfer in Mouse Intestine

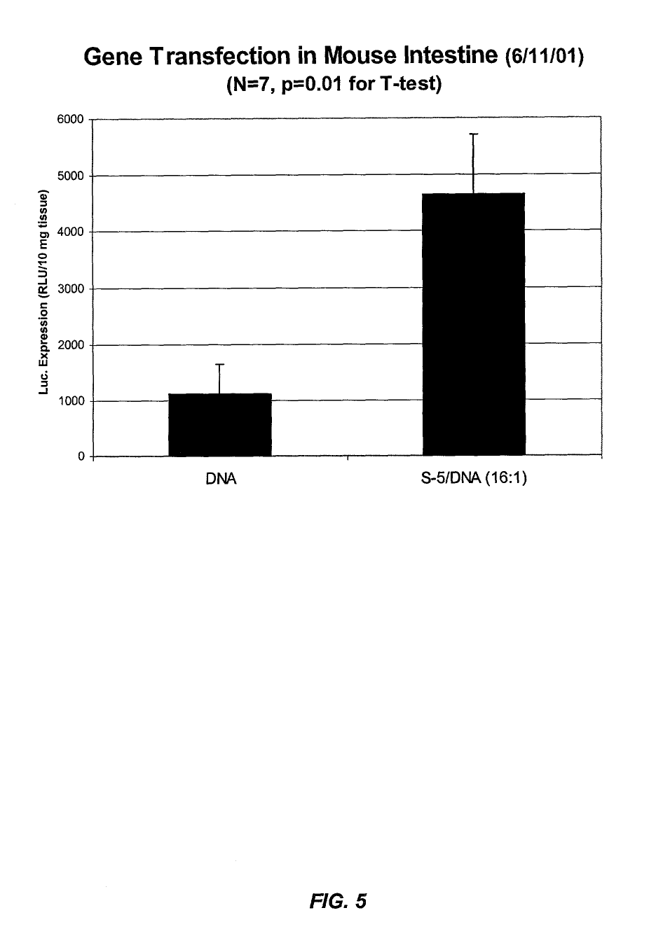

Luciferase gene solution (0.5 mg/mL, 1.8 mL) was added into the solution of S-5 (48.48 mM, 1.8 mL, containing 20 mM pH 7.4 HEPES buffer), and vortex mixed for 15 sec. DNA solution (0.5 mg/mL, 1.8 mL) was mixed with water (1.8 mL) to give the solution as the positive control. Male BALB/c mice (specific pathogen free, Harlan Co. CA) weighting 17-20 g were used. Animals were fasted overnight (water ad lib). After anesthesia with Isoflurane, a midline laparotomy was performed. The duodenum was exposed through the incision. The intestine was pretreated with Mucomyst. At 2 cm below pylorus, a solution of Mucomyst (10%, 300 μl) was injected in the duodenum. A 2 minutes, a DNA solution (400 μl) was injected into the duodenum. The animals were euthanized 24 hrs after the treatment. The duodenum and jejunoileum were removed separately. All intestinal tissues were homogenized in cold lysis buffer (0.1 mL buffer per 0.1 g tissue). The gene expression was determined by reading with a Luciferase Luminometer, and is set forth in FIG. 5.

### D. Example 4

Protocol for Gene Transfer in CHO Cells

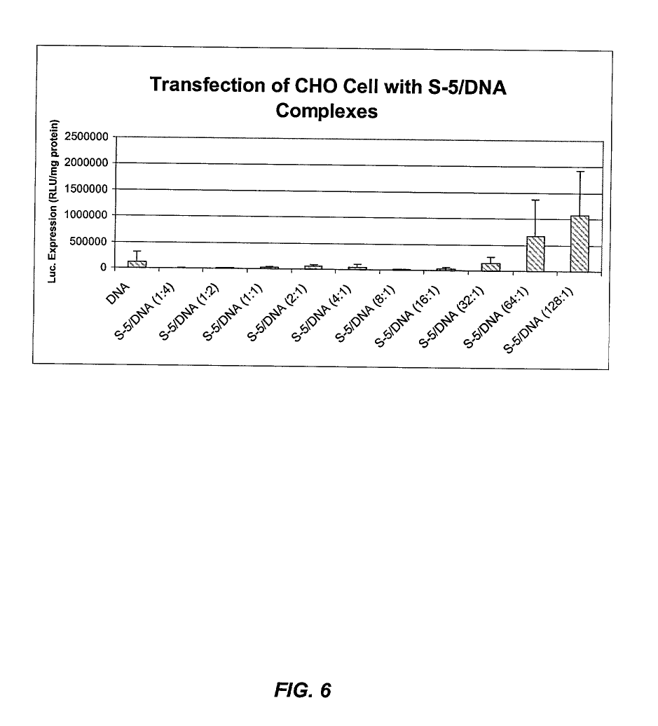

CHO cells 1.75×10⁴ per well was seeded in 24 well plate, and incubated in 10% serum DMEM media at 37° C. under the presence of 5% CO₂ for 48 h. The medium was removed, and washed twice with serum free DMEM medium. The DNA solution 200 μl was added per well (1 μg DNA per well). After incubation at 37° C. for 4 hours, wash once with DMEM media, and add DMEM+10% FCS (0.5 mL) followed by incubation for 48 h. The medium was removed, and the cells were washed with PBS twice and harvest with Luciferase Lysis Buffer. The gene expression was determined by reading with the Luciferase Luninometer, and is set forth in FIG. 6.

Cationic lipid carriers have been shown to mediated intracellular delivery of plasmid DNA (Felgner et al., 1987, Proc. Natl. Acad. Sci (USA), 84:7413-7416); mRNA (Malone et al., 1989, Proc. Natl. Acad. Sci. (USA) 86:6077-6081); and purified transfection factors (Debs et al., 1990, J. Biol. Chem. 265:10189-10192, in functional form. Literature describing the use of cationic lipids as DNA carriers included the following: Zhu et al., 1993, Science, 261:209-211; Vigneron et al., 1996, Proc. Natl. Acad. Sci. (USA) 93:9682-9686; Hofland et al., 1996, Proc. Natl. Acad. Sci (USA), 93:7305-7309; Alton et al, 1993, Nat. Genet. 5:135-142; von derLeyen et al., 1995, Proc. Natl. Acad. Sci. (USA), 92:1137-1141. For a review of liposomes in gene therapy, see Lasic and Templeton, 1996, Adv. Drug Deliv. Rev. 20: 221-266.

The role of sugar domain in targeted drug/DNA delivery is described in Wu et al., J Biol Chem. 1988 Apr 5;263(10):4719-23; Molema et al., Biochem Pharmacol. 1990 Dec 15;40(12):2603-10; Seymour et al., Br J Cancer, 1991 Jun; 63(6):859-66; Haensler et al., Bioconjug Chem. 1993 Jan-Feb;4(1):85-93; Nishikawa et al., Pharm Res. 1993 Sep;10(9):1253-61; Gonsho et al., Biol Pharm Bull. 1994 Feb;17(2):275-82; Martinez-Fong et al., Hepatology. 1994 Dec;20(6):1602-8; Nishikawa et al., Pharm Res. 1995 Feb;12(2):209-14; Zanta et al., Bioconjug Chem. 1997 Nov-Dec;8(6):839-44; Jager et al., Gene Ther. 1999 Jun;6(6):1073-83; Matsuura et al., Bioconjug Chem. 2000 Mar-Apr;11(2):202-11; Nishikawa et al., Gene Ther. 2000 Apr;7(7):548-55; Singh etal., Drug Deliv. 2001 Jan-Mar;8(1):29-34.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound having Formula I 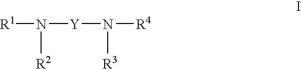
wherein,
R¹ is a C₃-C₂₀ carbohydrate with an optional linker;
R² is a member selected from the group consisting of a hydrogen, an alkyl group, and a boronic acid group;
Y is an optionally substituted alkylene group or —(CH₂CH₂O)ₘ—, wherein m is about 2 to about 80;
R³ is a member selected from the group consisting of a hydrogen, an alkyl group, and a cationic moiety; and
R⁴ is a member selected from the group consisting of an alkyl group, a cationic moiety; or alternatively,
R³ and R⁴ and the nitrogens to which they are attached, join together to form an optionally substituted five- or six-membered carbocyclic or heterocylic ring.

2. The compound according to claim 1, said compound having the formula: 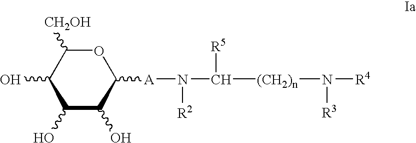
wherein:
A is a linker or C₆-C₁₂ carbohydrate with an optional linker;
R² is a member selected from the group consisting of a hydrogen, an alkyl group, and a boronic acid group;
n is an integer from 1-5 inclusive;
R³ is a member selected from the group consisting of a hydrogen, an alkyl group, and a cationic moiety; and
R⁴ is a member selected from the group consisting of an alkyl group, a cationic moiety; or alternatively,
R³ and R⁴ and the nitrogens to which they are attached, join together to form an optionally substituted five- or six-membered carbocyclic or heterocylic ring.

3. The compound according to claim 2, wherein said compound has the formula: 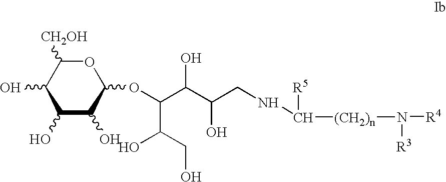
wherein:
R³ is a member selected from the group consisting of a hydrogen, an alkyl group, and a cationic moiety; and
R⁴ is a member selected from the group consisting of an alkyl group, a cationic moiety; and
R⁵ is a member selected from the group consisting of a hydrogen, a carboxyl group and an alkyl group; and
n is 2-3; or alternatively,
R³ and R⁴ and the nitrogens to which they are attached, join together to form an optionally substituted five- or six-membered carbocyclic or heterocylic ring.

4. The compound according to claim 3, wherein
R³ is an alkyl group; and
R⁴ is an alkyl group.

5. The compound according to claim 4, wherein said compound is selected from the group consisting of

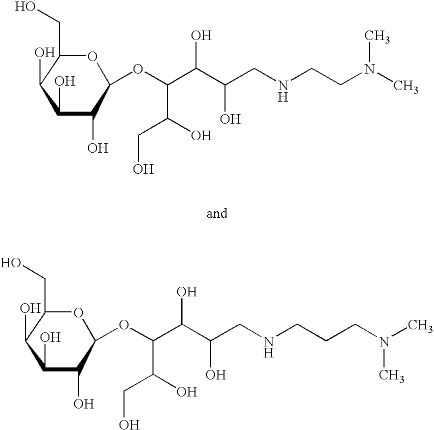

6. The compound according to claim 3, wherein
R³ is hydrogen; and
R⁴ is a carbamimidoylamino group or a salt thereof.

7. The compound according to claim 3, wherein
R³ and R⁴ and the nitrogens to which they are attached, join together to form a substituted heterocylic ring.

8. The compound according to claim 7, wherein said compound is selected form the group consisting of

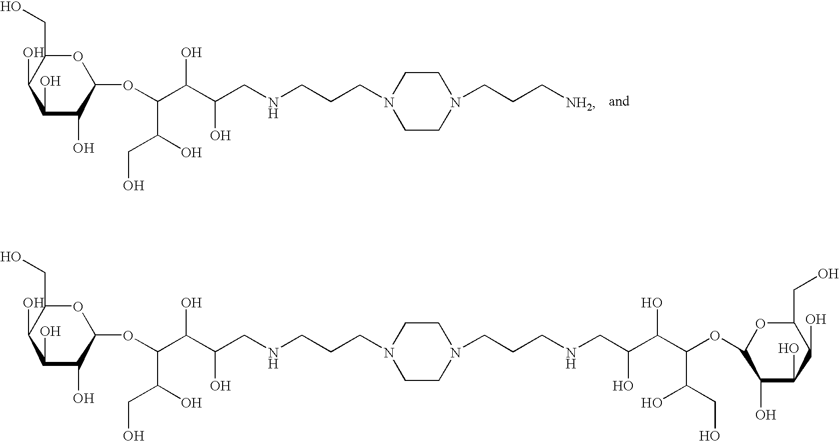

9. The compound according to claim 3, wherein
R³ is a guanidinoalkyl group or salt thereof; and
R⁴ is a guanidinoalkyl group or salt thereof.

10. The compound according to claim 9, wherein said compound is selected form the group consisting of

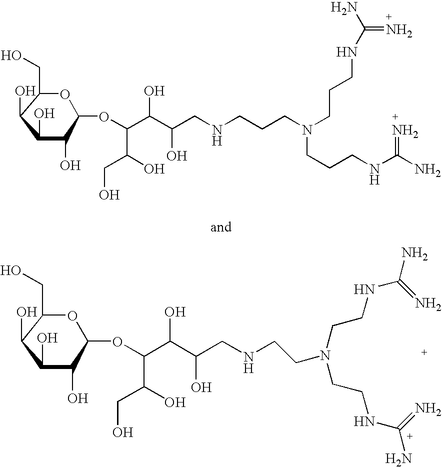

11. A transfection complex comprising a nucleic acid and a compound having Formula I: 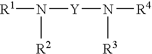
wherein:
R¹ is a C₃-C₂₀ carbohydrate with an optional linker;
R² is a member selected from the group consisting of a hydrogen, an alkyl group, and a boronic acid group;
Y is an optionally substituted alkylene group or —(CH₂CH₂O)ₘ—, wherein m is about 2 to about 80;
R³ is a member selected from the group consisting of a hydrogen, an alkyl group, and a cationic moiety; and
R⁴is a member selected from the group consisting of an alkyl group, a cationic moiety; or alternatively,
R³ and R⁴ and the nitrogens to which they are attached, join together to form an optionally substituted five- or six-membered carbocyclic or heterocylic ring.

12. The transfection complex according to claim 11, said compound having the formula: 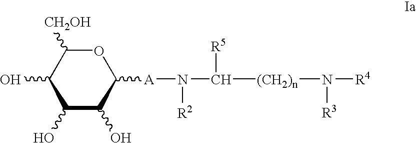
wherein:
A is a linker or C₆-C₁₂ carbohydrate with an optional linker;
R² is a member selected from the group consisting of a hydrogen, an alkyl group, and a boronic acid group;
n is an integer from 1-5 inclusive;
R³ is a member selected from the group consisting of a hydrogen, an alkyl group, and a cationic moiety; and
R⁴ is a member selected from the group consisting of an alkyl group, a cationic moiety; or alternatively,
R³ and R⁴ and the nitrogens to which they are attached, join together to form an optionally substituted five- or six-membered carbocyclic or heterocylic ring.

13. The transfection complex according to claim 12, wherein said compound has the formula: 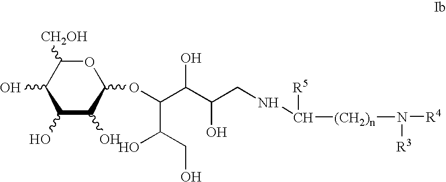
wherein:
R³ is a member selected from the group consisting of a hydrogen, an alkyl group, and a cationic moiety; and
R⁴ is a member selected from the group consisting of an alkyl group, a cationic moiety; and
R⁵ is a member selected from the group consisting of a hydrogen, a carboxyl group and an alkyl group; and
n is 2-3; or alternatively,
R³ and R⁴ and the nitrogens to which they are attached, join together to form an optionally substituted five- or six-membered carbocyclic or heterocylic ring.

14. The transfection complex of claim 11, wherein said nucleic acid is plasmid DNA.

15. The transfection complex of claim 11, wherein said nucleic acid is antisense RNA or DNA.

16. A method for transfecting mammalian cells, said method comprising contacting a nucleic acid with a compound having Formula I 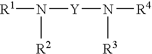
wherein:
R¹ is a C₃-C₂₀ carbohydrate with an optional linker;
R² is a member selected from the group consisting of a hydrogen, an alkyl group, and a boronic acid group;
Y is an optionally substituted alkylene group or —(CH₂CH₂O)ₘ—, wherein m is about 2 to about 80;
R³ is a member selected from the group consisting of a hydrogen, an alkyl group, and a cationic moiety; and
R⁴ is a member selected from the group consisting of an alkyl group, a cationic moiety; or alternatively,
R³ and R⁴ and the nitrogens to which they are attached, join together to form an optionally substituted five- or six-membered carbocyclic or heterocylic ring.

17. The method according to claim 16, said compound having the formula: 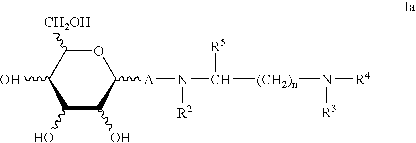
wherein:
A is a linker or C₆-C₁₂ carbohydrate with an optional linker;
R² is a member selected from the group consisting of a hydrogen, an alkyl group, and a boronic acid group;
n is an integer from 1-5 inclusive;
R³ is a member selected from the group consisting of a hydrogen, an alkyl group, and a cationic moiety; and
R⁴ is a member selected from the group consisting of an alkyl group, a cationic moiety; or alternatively,
R³ and R⁴ and the nitrogens to which they are attached, join together to form an optionally substituted five- or six-membered carbocyclic or heterocylic ring.

18. The method according to claim 17, wherein said compound has the formula: 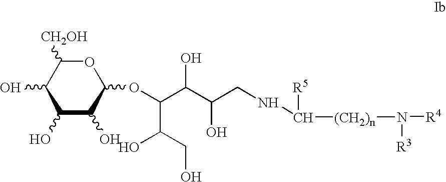
wherein:
R³ is a member selected from the group consisting of a hydrogen, an alkyl group, and a cationic moiety; and
R⁴ is a member selected from the group consisting of an alkyl group, a cationic moiety; and
R⁵ is a member selected from the group consisting of a hydrogen, a carboxyl group and an alkyl group; and
n is 2-3; or alternatively,
R³ and R⁴ and the nitrogens to which they are attached, join together to form an optionally substituted five- or six-membered carbocyclic or heterocylic ring.

19. The method according to claim 16, wherein said contacting is performed in vitro.

20. The method according to claim 16, wherein said contacting is performed in vivo.

21. The method according to claim 16, wherein said contacting is performed by oral administration.

22. The method according to claim 16, wherein said contacting is performed by retrograde induction.

23. The method according to claim 16, wherein said nucleic acid is plasmid DNA.

24. The method according to claim 16, wherein said nucleic acid is antisense DNA or RNA.
